(19) United States
(12) Patent Application Publication
(10) Pub. No.: US 2004/0253168 A1
(43) Pub. Date: Dec. 16, 2004

(54) System and method for hydrocarbon processing

(76) Inventor: Xi Chu, Mounds View, MN (US)

Correspondence Address: Xi Chu, 5273 Sunnyside Rd, Mounds View, MN, 55112, US

(21) Appl. No.: 10/830,594

(22) Filed: Apr. 23, 2004

Related U.S. Application Data
(60) Provisional application No. 60/464,386, filed on Apr. 23, 2003.

(51) Int. Cl. C01B 31/02; B32B 5/02; F28D 1/00; G01N 33/00; G01N 27/00

(52) U.S. Cl. 423/447.3; 422/188; 422/198; 422/83; 422/186; 422/186.04

(57) ABSTRACT

This patent discloses a system and method of producing nanostructured carbon and carbon monoxide-free hydrogen through the decomposition of hydrocarbons in a spouted bed reactor. The process is precisely controlled in such a way that the carbon particles generated in reaction has a unique nanostructure so their surfaces can act as catalytic sites for the decomposition of hydrocarbons. The process produces hydrogen stream containing no carbon monoxide, and The CO-free hydrogen is ideal fuel for fuel cells (especially the PEM) and many industrial chemical syntheses. The generated nanostructured carbon can be used as catalyst for the processing of hydrocarbons such as hydrogenation, dehydrogenation and partial oxidation of hydrocarbon chemicals. In addition, the nanostructured carbon produced can be used as electrode material for electrochemical energy conversation and storage and industrial electrochemical processes, fuel for the direct carbon fuel cell, and fillers of medical implants and components.

### RELATED APPLICATION

The present application claims priority under 35 U.S.C. §119(e) to U.S. Ser. No. 60/464,386 filed on Apr. 23, 2003, the entire content of which is incorporated herein by reference.

### GOVERNMENT INTERESTS

The Government of the United States of America has rights in this invention pursuant to Grant No. 0231107 awarded by the National Science Foundation, Grant No. DE-FG02-04ER84084 awarded by the U.S. Department of Energy, and Grant No. 53120A/02-21. by California Energy Commission

### FIELD OF THE INVENTION

The present invention generally relates to a method and system of producing nanostructured carbon from hydrocarbons and use of nanostructured carbon as catalyst to carry out the desired chemical reactions. The processes are particularly but not exclusively directed to the hydrogen and carbon productions, hydrogenation and partial oxidations of chemicals in gas or liquid phase reactions where conventional metal or oxide catalysts are required. In addition to be used as catalyst, the nanostructured carbon can be used as electrode material in electrochemical cells and reactions, and fillers of medical implants and components.

### BACKGROUND OF THE INVENTION

Hydrocarbon processing (treating hydrogen and carbon containing chemicals in chemical reactions to make new products) has many industrial applications. Examples are the industrial hydrogen production (used for the fertilizer production and oil reforming), petroleum processing, hydrogenation and partial oxidation of hydrocarbons, etc.

The discussion below will be focused on nanostructured carbon catalyzed CO-free hydrogen production from hydrocarbons for fuel cell application. This will be used as an example of hydrocarbon processing because many other hydrocarbon reactions such as hydrogenation, dehydrogenation and partial oxidation etc. are parallel in nature and can be directly applied.

Hydrogen is a critical raw material for many industrial processes. Currently, hydrocarbon steam reforming to form syngas is the primary industrial step for hydrogen production. It is very energy and capital intensive, operating at high pressure and temperatures. In addition to the energy for the reactions, other energy required includes feedstock treatments and steam production. It consumes a significant amount of hydrocarbon feedstock as process fuel. Furthermore, the process generates huge amount of green house gas carbon dioxide making the carbon sequestration another environmental challenge.

Fuel cells represent the next generation energy technologies due to their high energy conversion efficiency. Among all fuel cell technologies, the proton exchange membrane (PEM) fuel cell has received the most attention because it has many advantages such as low temperature operation, simple design, high power density, long use life, and pressure insensitivity compared with other fuel cell technologies. However, the platinum catalyst used in the PEM fuel cell can be poisoned by impurities especially carbon monoxide (CO) even as low as a few parts per million (PPM). The presence of CO in reformate streams is almost unavoidable, even with the state-of the-art reforming technology, because the involved processing steps such as steam reforming, partial oxidation, and water-gas shift reaction all generate or have residue of carbon monoxide in the hydrogen stream due to the thermodynamic equilibrium limits. Even though multiple technologies and processes have been explored, an effective and affordable hydrogen production and purification system for PEM fuel cell is still unavailable.

Furthermore, conventional hydrogen production is highly centralized. The distribution and storage of hydrogen is extremely challenging. The dynamic need and flexible on-site production is the primary requirement. Since there is a natural gas and gasoline distribution infrastructure and they are abundant hydrogen sources, in a recently published U.S. DOE Hydrogen Posture Plan, CH₄ is the near term option for the H₂ and this allows on site production to avoid the distribution and storage hurdles.

1. Thermal Decomposition of Hydrocarbons

Hydrogen can be generated through thermal cracking of hydrocarbons such as CH₄, C₃H₈ and petroleum. The product stream is free of carbon oxides. For example, thermal decomposition of natural gas has been practiced for decades as a means of producing carbon black with hydrogen being a supplementary fuel for the process (Thermal Black process). U.S. Pat. Nos. 5,859,120; 5,891,414; 5,914,093; 6,068;827; 6,096,284; 6,132,876; 6,136,286; 6,358,487; 6,391,274; 6,548,036; 6,652,641, et al taught the different methods of producing carbon blacks from hydrocarbons. In these processes, hydrocarbon stream was pyrolyzed at high temperature (over 1400° C.) by partial combustion of the hydrocarbons and water quenching to prevent the reverse reaction. This causes the contamination of the hydrogen stream. In addition, the efficiency and the yield are extremely low. Another challenge in hydrocarbon decomposition is that it is difficult to handle the carbon build up on a continuous basis.

Kvaemer Company of Norway has developed a methane decomposition process which produces hydrogen and carbon black by using high temperature plasma (U.S. Pat. No.5,527,518). The advantages of the plasmochemical process are high thermal efficiency (>90%) and purity of hydrogen (98 v. %), however, it is an electric energy intensive process.

Steinberg et al. proposed a methane decomposition reactor consisting of a molten metal bath (Int. J. Hydrogen Energy, 24, 771, 1999). Methane bubbles through molten tin or copper bath at high temperatures (900° C. and higher). The advantages of this system are: an efficient heat transfer to a methane gas stream, and, ease of carbon separation from the liquid metal surface by density difference.

2. Catalytic Thermal Decomposition of Hydrocarbons

There have been attempts to use catalysts to reduce the maximum temperature of the thermal decomposition of hydrocarbons. Common catalysts are noble and transitional metals such as Pt, Ru, Ir, Pd, Ni, Fe, Co etc. supported on high surface area ceramic substrates such as A1₂O₃ and SiO₂ etc. These catalysts are very expensive due to the material used and their preparation processes. In addition, the deactivation of the catalyst occurs immediately after the reaction due to “coking”, or carbon deposition on the metal catalysts that covers the catalytic active sites. This requires consistent regeneration of the catalyst by burning off the carbon deposite periodically, which causes the lose of the metal catalysts, reduce the lifetime and adds inconvenience and cost to the process operation and thus the cost of the final products. In addition, the regeneration of the catalysts causes the contamination of the stream with carbon oxides.

For example, U.S. Pat. No. 3,284,161 to Pohlenz et al. describes a process for continuous production of hydrogen by catalytic decomposition of a gaseous hydrocarbon streams. Methane decomposition was carried out in a fluidized bed catalytic reactor in the range of temperatures from 815 to 1093° C. Supported Ni, Fe and Co catalysts (preferably Ni/Al₂O₃) were used in the process. The coked catalyst was continuously removed from the reactor to the regeneration section where carbon was burned off, and the regenerated catalyst was recycled to the reactor.

U.S. Pat. No. 2,476,729 to Helmers et al. describes the improved method for catalytic cracking of hydrocarbon oils. It was suggested that air is added to the feedstock to partially combust the feed such that the heat supplied is uniformly distributed throughout the catalyst bed. This, however, would contaminate and dilute hydrogen with carbon oxides and nitrogen.

3. Carbon Involved Thermal Decomposition of Hydrocarbons

Several patents disclose the use of carbon-based materials for decomposition of hydrocarbons into hydrogen and carbon. It has also been taught to thermally decompose hydrocarbon feedstock over carbon particles acting as a heat carrier.

U.S. Pat. No. 2,805,177 to Krebs describes a process for producing hydrogen and product coke via contacting a heavy hydrocarbon oil admixed with a gaseous hydrocarbon with fluidized coke particles in a reaction zone at 927-2100 F. Gaseous products containing at least 70 v. % of hydrogen were separated from the coke, and a portion of coke particles was burnt to supply heat for the process; the remaining portion of coke was withdrawn as a product. About 1200 lbs of coke is circulated per mole of methane. The large amount of carbon particles and the strict size requirement makes it impractical for production.

U.S. Pat. No. 4,056,602 to Matovich deals with high temperature thermal reactions, including the decomposition of hydrocarbons, by utilizing fluid wall reactors. Thermal decomposition of methane was conducted at 1260-1871° C. using carbon black particles as adsorbents of high flux radiation energy, and initiators of the pyrolytic dissociation of methane.

U.S. Pat. No. 5,650,132 to Murata et al. produces hydrogen from methane and other hydrocarbons by contacting them with fine particles of a carbonaceous material obtained by arc discharge between carbon electrodes and having a large external surface area of at least 1 m₂/g. Carbonaceous materials also included: soot obtained from the thermal decomposition of different organic compounds or the combustion of fuels; carbon nanotubes; activated charcoal; fullerenes C₆₀ C₇₀; and, finely divided diamond. The optimal conditions for methane conversion included: methane dilution with an inert gas (preferable methane concentration: 0.8-5% by volume); A temperature range of 400-1,200° C.; and residence times of −50 sec. An increase in methane concentration in feedstock from 1.8 to 8 v. % resulted in a drastic drop in methane conversion from 64.6 to 9.7% (at 950° C.).

It was also stated that during hydrocarbon pyrolysis (the experiments usually ran for 30 min) the carbon samples gradually lost their catalytic activity. It was suggested that oxidizing gases like H₂O or CO₂ be added to the pyrolyzing zone to improve the catalyst life. However, this would inevitably contaminate hydrogen with carbon oxides and require an additional purification step. Also, it was suggested that the spent catalyst be combusted, which would be, however, very wasteful, especially, considering the high cost of the carbon materials used in the process. Therefore, no application is visualized for this technique.

U.S. Pat. No. 6,670,058 to Muradov discloses a process for CO₂-free production of hydrogen and carbon by thermochemical decomposition (or dissociation, pyrolysis, cracking) of hydrocarbon fuels over carbon-based materials in the absence of air and/or water. Combination of the reactor with a gas separation unit allows to produce high purity hydrogen (at least, 99.0 v %) completely free of carbon oxides.

This process was operating at a low temperature (T<800° C.) and very low rate has been reported. It relied on high surface area carbon particles such as carbon black, activated carbon or even ceramic powders. Once the initial surface is covered by carbon deposite, both internal and external activation of carbon catalysts are required to restore the activity. Internal activation of carbon is suggested by recycling of hydrogen-depleted gas containing unsaturated and aromatic hydrocarbons back to the reactor. External activation can be achieved via surface gasification of carbon particles by hot combustion gases during heating, and these are similar to the treatment of conventional supported catalysts in hydrocarbon processings with heavy cross contamination.

In summary of the foregoing, the major problem with the decomposition of methane (or other hydrocarbons) over carbon (or any other) catalysts relates to their gradual deactivation during the process. This could be attributed to two major factors: (i) loss of active surface area; and, (ii) inhibition of the catalytic process by the deposition of carbon species which are less catalytically active than the original catalyst. In addition, carbon also deposits around the interior wall of the reactor in all the processes, and this gradually decreases or even blocks the passway of the reactor systems. In all the patents cited above, this basic fact has been avoided by the inventors either their process had not been operated long enough (most only shows 30 min operation) or was intended avoided. Thus, the need exists for a more effective, versatile and cost effective process for CO-free production of hydrogen and carbon from wide range of hydrocarbons using inexpensive and readily available process.

U.S. Pat. No. 5,874,166 to Chu and Kinoshita demonstrates that the catalytic properties of carbonaceous materials are determined by their structures. Only the edges of the graphitic domains are catalytic sites while the basal plane is inert to chemical reaction. On the other hand, the structure of carbon can be precisely controlled in a spouted bed chemical reactor as demonstrated in the application for the nuclear fuel coating. Therefore, the present invention is directed to overcome the difficulties mentioned above through the following approaches: First, precisely control the process conditions so the carbon generated has a unique surface structure composing of catalytic sites and further deposition only add more catalytic site without changing the structure. Second, add small particles and withdraw large carbon particles during operation to balance the deposition condition, namely the total surface area. Finally, recondition the internal wall of the reactor periodically to remove the carbon built up and to ensure continuous operation.

### SUMMARY OF THE INVENTION

The present invention relates to generally relates to the processing of hydrocarbon chemicals using nanostructured carbon as catalyst and a method and system of processing the catalyst and carrying out the desired reaction pathway, thus the right products of the same. To produce the nanostructured carbon catalyst, hydrocarbons are decomposed in and catalyzed by nanostructured carbon itself in a spouted bed chemical reactor. The processes are particularly but not exclusively directed to the hydrogen and carbon productions, hydrogenation, dehydrogenation, and partial oxidations of chemicals in gas or liquid phase reactions.

In this process, small carbon catalyst particles (50-2000 microns) are introduced into the reactor as catalyst to provide surface catalytic sites for hydrocarbon decomposition and carbon deposition. Most importantly, the process is controlled in such a way that solid carbon deposited on the surface of the particles is unique in structure; it is isotropic at micrometer scale with nanometer size graphitized domains randomly orientated so all the newly generated surfaces are active catalytic sites for the decomposition (edge sites are catalytic!). This ensures a high rate and stable reaction. Meanwhile large solid carbon particles are continuously withdrawn from the reactor to balance the total surface areas within the reactor chamber and ensure proper carbon structure. The large carbon particles can be ground to smaller particles to be used as feed material in this, or as catalyst in other hydrocarbon processing such as hydrogenation, dehydrogenation, and partial oxidations, etc. Furthermore, the internal carbon built up will be removed periodically through an integrated device. In addition to be as catalysts, the nanostructured carbon can be used as electrode materials and fuel of electrochemical reaction and deice. Furthermore, it can be used as filler in medical implants and components.

Process Advantages

The present invention further provides an improved hydrogen production process that is energy saving and environmental benign. Compared with conventional steam reforming, this approach has the following advantages:

1. Low capital cost since no expensive catalysts and no large capital equipment are involved.

2. High rate, small reactor, and high space velocity

3. Long life and low maintenance cost because of self-generation of catalytic activity

4. The product stream contains only H₂ and a small fraction of light hydrocarbons, and therefore the separation process is relatively simple by common practice.

5. No carbon monoxide (CO) in the product hydrogen stream, so it is the ideal fuel for PEM fuel cell (PEM fuel cell catalyst deactivation issue is completely eliminated).

6. The nanostructured carbon can be used as catalysts for other hydrocarbon processings or be used in carbon fuel cell, batteries, and electrolysis industries. The carbon black (current annual production is several billion kilograms) can be used in the rubber and plastic industries.

7. The process generates little or even no CO₂ compared with conventional fuel reforming to obtain H₂. Thus the extremely expensive CO₂ sequestration should be of less concern.

The present invention provides the novel nanostructured carbon catalyst, reactor designs, manufacturing method, and the integration of the systems that ensures improved performance of chemical reaction and other applications. Specifically, The present invention further provides an improved hydrogen production process that is free of CO, energy saving and environmental benign.

In the preferred embodiment of the invention is the carbon particles generated are nanostructurely engineered in such a way that all the graphitic domains are preferred aligned perpendicular to the surface through the control of the coating parameters. The surface of the final particle consists of the edge sites of graphite domains. Therefore the catalytic activities of the carbon particles can be greatly enhanced.

One preferred embodiment relates to a method for producing nanostructured carbon and hydrogen from hydrocarbons comprising the steps of: decomposing hydrocarbons thermally using a first carbon particles as substrates and catalysts in a reactor; removing a hydrogen-containing gas from the reactor; separating hydrogen from the hydrogen-containing gas; and withdrawing a second carbon particles from the reactor. The method also includes the step of grinding certain amount of the second carbon particles periodically; and reintroducing into the reactor to balance the total bed surface area.

Another preferred embodiment relates apparatus for hydrocarbon processing, the apparatus comprising: a plurality of spout bed chambers; a heating system; a thermal insulation system; a chemical introducing system; a gas and solid separation system; a gas analysis system; a plurality of introducing ports; a particle feeding system; a particle withdrawing system; an internal grinding system; a preheating and heat recovery system; and a monitor system for the structure of carbon particles. The heating system is chosen from the group comprising: electrical resistive heating; RF inducting heating; thermal plasma heating; combustion heating by a self-heating using hydrogen, un-reacted hydrocarbon, carbon particles, or other fuels; solar energy; and nuclear energy heating.

Another preferred embodiment relates to a composition of nanostructured carbon, characterized in that comprising: a density from 1.7 g/cc to 2.3 g/cc; a lattice spacing from 2.37 A to 2.8 A; a crystalline size from 10 A to 200 A; The nanostructured carbon can be used as catalyst in hydrocarbon reactions, including decomposition of hydrocarbons, partial oxidation of hydrocarbons, hydrogenation of hydrocarbons, and dehydrogenation of hydrocarbons. The nanostructured carbon can be used as solid fuel of direct carbon fuel cells, as anode of lithium ion battery, in an electrochemical device and as fillers or components of an implantable medical device.

### BRIEF DESCRIPTION OF THE DRAWINGS

The drawings form part of the present specification and are included to further demonstrate certain aspects of the present invention. The invention may be better understood by reference to one or more of these drawings in combination with the detailed description of specific embodiments presented herein.

FIG. 1 Process flow of the spouted bed reactor for the continuous thermal decomposition of hydrocarbons

FIG. 2 Diagram of the spouted bed reactor system used in this invention

FIG. 3a Schematic arrangements of the spouted bed reactor system

FIG. 3b Schematic arrangements of the spouted bed reactor system with multiple spouting ports

FIG. 3c Schematic arrangements of the spouted bed reactor system with RF induction heating

FIG. 4a Schematic structure of nanostructured carbon.

FIG. 4b High resolution transmission electron micrograph of nanostructured carbon.

FIGS. 4c & d Scanning electron micrographs of the cross sections of nanostructred carbon particles embedded in epoxy resin for property evaluation

FIG. 5 Optical micrographs of the cross sections of nanostructred carbon particles embedded in epoxy resin for evaluation under polarized light for aniostripic properties evaluation.

### DETAILED DESCRIPTION OF THE INVENTION

The present invention relates to production of a nanostructured carbon and processing of hydrocarbons using the nanostructured carbon as catalyst in a spouted bed chemical reactor. The specific reactions include but not limited to hydrogenation, dehydrogenation, and partial oxidation.

Reactor Design and Process

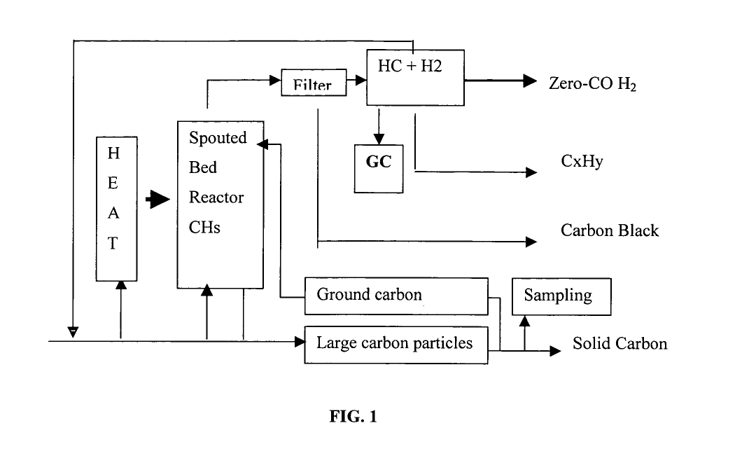

FIG. 1 shows the process flow of the reactor system in this invention. It was used for the nanostructured carbon generation and other catalytic reactions using the nanostructured carbon catalyst generated for hydrocarbon processing. The process rate and the structure of the carbon are determined by many factors such as process temperature, gas composition, flow rate or special velocity, carbon bed particle size and total volume or surface area of the carbon particles in the bed. Best conditions for individual reaction process with a particular reactor design and configuration can be identified by design of experiment per common engineering practice.

The reactor is electrically heated or by other options including a self-heating using H₂, un-reacted hydrocarbons, or even solar or nuclear heat to a temperature between 100 to 3000° C., preferred between 1000-1800° C. Hydrocarbon chemicals (20-100 % hydrocarbons in N₂) are fed through the bottom of the reactor. The pressurized hydrocarbon chemicals (can be mixed with inert diluting gases, 0-1000 psi, such as N₂, Ar or He are premixed according to process design) are controlled using mass flow controllers.

Initial carbon particles (0.3-1.0 mm in diameter) are filled in the reactor to create a high surface area for the carbon decomposition. Small carbon particles (0.2-0.5 mm in diameter) are added to the reactor through the feeder and large carbon particle (0.2-5.0 mm in diameter) are withdrawn to the receiver. Most importantly, the process is controlled in such a way that solid carbon particles generated are unique in structure. They are isotropic carbon with all newly generated surfaces being active catalytic sites for the reaction and this ensures the high reaction rate and continuous reaction. Meanwhile large solid carbon particles are withdrawn from the reactor continuously to balance the total surface area within the reactor chamber and to ensure proper carbon structure.

The key of this process is to convert hydrocarbons into hydrogen and solid carbonaceous materials. Unlike the conventional industrial hydrogen generation using reforming and gas shift reaction, the hydrogen stream contains no carbon oxides. This will save the separation cost and allow fuel to be used for fuel cell application without complex CO removal processes. In addition, since the separation technique is easy to apply and the byproduct is value-added.

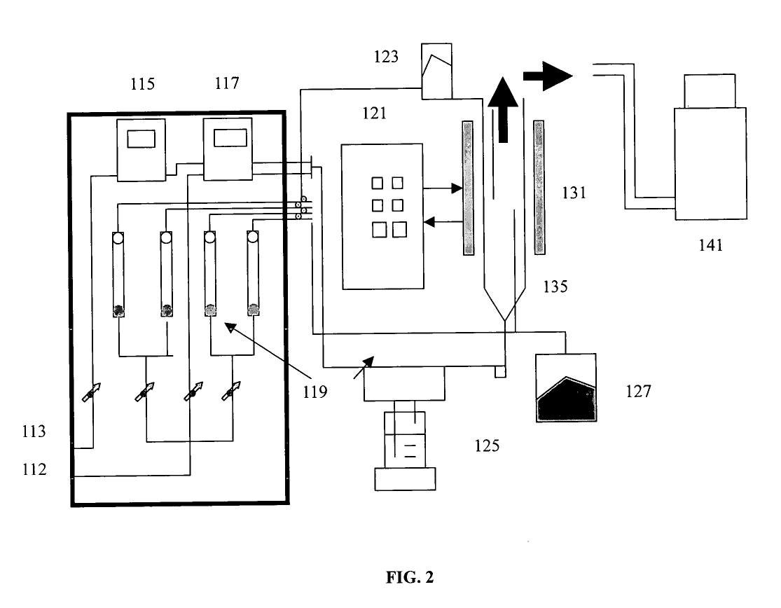

FIG. 2 illustrates the actual process apparatus that makes the nanostructurely engineered carbon material and the production of CO-free hydrogen. It consists of the following sub-systems:

a. The process gas mixing and delivery system

b. The reactor hardware, heating, and control system

c. The media withdraw and the particle feeding system

d. The catalysts introducing system

e. The exhaust control and treatment system

Hydrocarbons are chemicals containing hydrogen and carbon elements in the molecules such as natural gas (methane), ethane, propane, and petroleum, renewable fuels and synthetic oil, and biomass etc. They are in gas, liquid, or solid form at their normal stage. They or their combination can be used as the main source of carbon for its high carbon content, low cost, availability and ease to handle. Hydrocarbons are introduced through line 113 (For methane, technical grade >98%; propane 40 lbs tank, purity 95% with the rest of other alkanes and tracing amount of other organic compounds).

Nitrogen 112 was used as protecting and diluting gas. Since our process consumes a large amount of nitrogen for each run (at a flow rate of combined gas from 10 to 100 l/min.), industrial liquid nitrogen was used (99.9%, 700 lbs tank containing about 30,000 liters of nitrogen gas).

Both hydrocarbons and nitrogen were controlled by separate mass flow controllers 115, 117 (Davis Instrument, which control flow rate 0-50 l/min with an accuracy of 0.5% at room temperature. The mass flow controller allows the setting of the ratio of the gases and the total flow rate for each run. In addition, as shown on the panel 119, nitrogen was also used to purge the system during heating up and cooling down of the reactor, to control the media withdraw from the reactor during the operation, and to control (through bubbling, as will be discussed in the catalysts introducing section) and delivery catalyst to the reactor.

The system has a custom made 20 kW electrical furnaces 131 that can be operated up to 1600° C. The furnace has 12 SiC electrodes connected in series and operated at 240V AC. It allows the heating from room temperature to the reaction temperature, normally 1300° C. within 30 min. The temperature can be controlled within 1.0° C. through a digital double feedback loop controller 121.

The reactor tube 135 is made of either graphite or fussed quartz. Attempt of making ceramic reactor components was also made. The reactor tube has a diameter of 75 mm and a wall thickness of 2.5 mm. Its bottom is a funnel shaped with a taping angle of 40 degrees. The bottom is connected with a thin tube with an ID of 6 mm and OD of 10 mm. This thin tube is connected with processing gas line after the mass flow controllers. The small diameter inlet allows the incoming gas to create a jet within the bottom of the reactor during the reaction, therefore, moving the media and the parts within the reacting chamber of the reactor to allow the deposition of carbon on all the surfaces of the parts and media particles.

During the manufacturing process, carbon deposits on all the surfaces including the media particles. Therefore, the volume of the media increases over time. The total surface area also increases as the parts and media particles grow. To maintain the consistent process condition thus good properties, large carbon media particles were withdrawn through the side port (connected with a container in a seal system with nitrogen purge all the time) of the reactor at the bottom 127. The amount of withdraw was controlled by nitrogen pressure through solenoid valves. At the same time, small carbon particles were fed at a consistent rate of 0.5 g/min from the top feeder 123 of the reactor to balance the total reactor bed material (media) volume and the surface area. The carbon media (initially loaded in the reactor) was prepared by grinding large PYC particles from the previous run and sieved to the size between 300 and 850 microns. The particles for the feeder 123 (feed into the reactor during run) were in the size range of 300-500 microns.

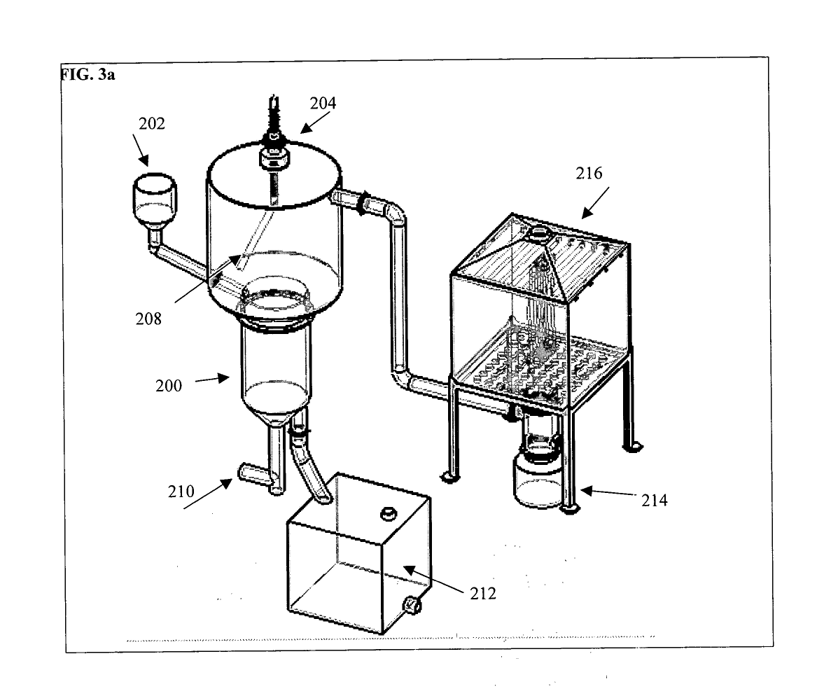

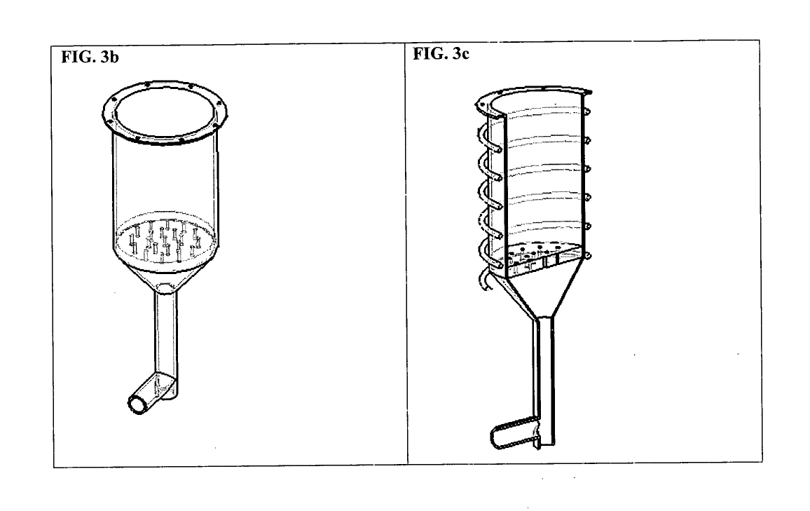

FIG. 3a is the schematic arrangement of the spouted-bed chemical reactor assembly. Processing gas enters the bottom of the reactor 210 to be decomposed in the reactor chamber 200. During the carbon preparation or the hydrocarbon decomposition cases, small carbon particles will be added through feeder 202 and large particles will be withdrawn to receiver 212. The internal wall of the reactor will be ground by the grinding stick 208, which is driven by the motor on the top of the reactor. The angle between the bars can be adjusted so the tip can reach all portion of the reactor internal wall. The product stream containing carbon black will enter the baghouse 216 so the solid can be separated from the stream and stored in the collector 214, and will be removed periodically.

FIG. 3b is the embodiment of a large reactor chamber with multiple spouting ports. This can be used for large-scale industrial production. FIG. 3c is a preferred embodiment with a radio frequency inducting heating system. In addition, to electrical resistive heating and the RF-induction heating, other embodiments for the heating can be plasma, solar, combustion using raw fuel, product hydrogen or carbon, and even nuclear heat.

In a preferred embodiment, the process gas can be pass through the RF coil to take the heat and preheat the gas to facilitate the reaction. In another embodiment, a heat exchanger can be installed to use the heat carried by the product gas for the preheat of the process gases to facilitate the reaction and reduce process energy consumption.

Structure and Catalytic Activities of Nanostructured Carbon Particles

Unlike other element, carbon has a wide range of structures corresponding to complete different properties. For examples, chemically soot, charcoal, graphite, and diamond are all made of carbon. However, their physical and chemical properties are quite different. Since the structure of the carbon has a great effect on the catalytic activities, the structures of the carbon generated were studied using high resolution transmission electron microscopy (TEM), scanning electron microscopy (SEM), optical microscopy and X-ray diffraction to gain atomic scale structure information. In addition, various phases of carbon can be distinguished using polarized optical microscopy. The structure of the carbon is a quality and process monitoring parameter. The nanostructured carbon generated through this invention has at least the following characteristics:

a density from 1.7 g/cc to 2.3 g/cc;

a lattice spacing from 2.37 A to 2.8 A; and

a crystalline size from 10-500 A

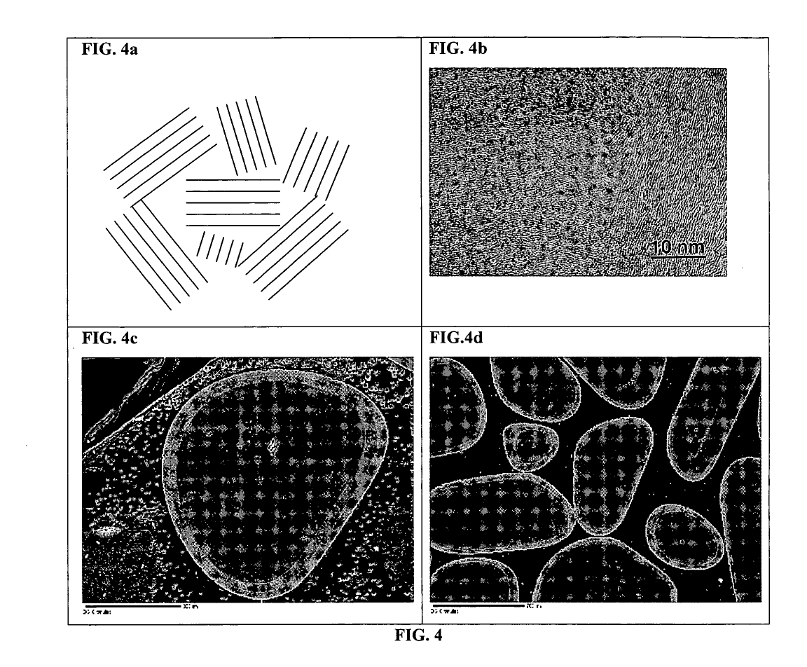

FIG. 4a is the schematic structure of nanostructured carbon produced by our process and FIG. 4b is a high resolution transmission electron micrograph of nanostructured carbon. This is an example of the high resolution structure of the nanostructured carbon material. It consists of many nanometer size domains and these domains are randomly orientated to form a solid dense structure. This is the preferred structure of the nanostructured carbon catalysts for our processes; the surfaces of the particles are highly active catalytic sites for carbon related reactions.

To monitor the process, the generated particles will be metallurgically mounted, sectioned and polished to get optical finish. They samples were then examined under polarized microscope to identify the microstructures. FIGS. 4c & d are scanning electron micrographs of the cross sections of nanostructrued carbon particles embedded in epoxy resin met mount for evaluation and for properties evaluation.

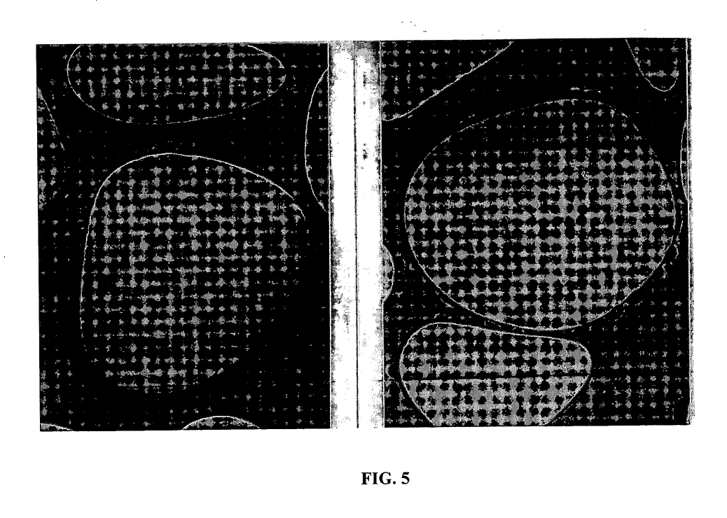

FIG. 5 shows optical micrographs of the cross sections of nanostructured carbon particles embedded in epoxy resin for evaluation under polarized light for aniostripic properties evaluation. Small particles inside large particles are evident. This was caused by our process nature that small particles are added into the reactor during the reaction, and once they were covered by carbon to become large particles, they were withdrawn from the reactor resulting a multilayer or inclusion structure.

The unique structure and properties of the nanostructured carbon make its good candidates as the fuel of direct carbon fuel cell, electrode materials of electrochemical cells and devices, and medical implant fillers or components.

Process Mornitoring: Conversion and Selectivity

With a given reactor design and size, the temperature distribution, the gas composition, and flow rate, and the bed surface area are the most important parameters in determining the carbon structure of the produced carbon particles. The amount of the carbon formation, the composition of the product stream is closely monitored to calculate the conversion and the yield and related them to the reaction parameters.

### EXAMPLES

### Example 1

### Reaction With Natural Gas

In a typical case with natural gas (CH₄), the reactor is preheated to the desired temperature with flowing N₂ (from liquid nitrogen tank). The bed materials (200 to 700 g) are ground and sieved particles from the previous runs with a size between 500 -850 microns. The natural gas (CH₄), from tank along (T-sized, from Praxair, grade 2.0 or 1.3) with diluting gas nitrogen was regulated through two mass flow controllers. The inlet pressure is maintained at 30 psi and the amount of methane is monitored using the flow rate. The gas mixture (the concentration was determined by experiment design) was introduced into the reactor when the reactor reaches the desired temperature. Once the run time is reached, the reaction is stopped and the reactor is cooled to room temperature and break down to extract the products. Since the density of the sample has a great impact on the mechanical strength of the mechanical properties, therefore, it was used as initial measure to monitor the process. In addition, the dimension or weight of the samples, the weight of carbon media left in the reactor (the size of the fluidized bed), the weight of the media withdrawn was measured.

The process and sample information is summarized in Table 1 below a total of 12 runs were conducted. Table 1 shows the example runs conducted. Specific details of the experiment associated with each run. Pure pyrolytic carbon samples were prepared as controls for the properties comparison. Carbon nanofiber reinforced pyrolytic carbon coating samples were prepared to compare the micro-scale structures and the macroscopic properties respectively.

TABLE 1
Experimental conditions and conversion for natural gas (methane) pyrolysis

| | | Time | CH₄ | N₂ | Bed particle size | | CH₄ to C |
|---|---|---|---|---|---|---|---|
| Run # | T (° C.) | (min) | (LPM) | (LPM) | (μm) | Initial Bed (g) | (captured) |
| 1-2 | 1350 | 60 | 12 | 6 | 500-850 | 200 | 32% |
| 2-4 | 1350 | 35 | 18 | 0 | 500-850 | 200 | 59% |
| 3-5 | 1350 | 60 | 18 | 0 | 500-850 | 200 | 33% |
| 4-7 | 1350 | 60 | 18 | 0 | 500-850 | 380 | 50% |
| 5-9 | 1350 | 60 | 12 | 0 | 500-850 | 550 | 73% |
| 6-10 | 1350 | 60 | 18 | 0 | 500-1000 | 700 | 53.5% |
| 7-11 | 1350 | 60 | 12 | 0 | 500-850 | 500 | 75% |
| 8-12 | 1300 | 60 | 12 | 0 | 500-850 | 650 | 60% |
| 9-13 | 1350 | 120 | 12 | 0 | 500-850 | 876 | 74% |
| 10-14 | 1350 | 60 | 12 | 0 | 500-850 | 1296 | 84% |
| 11-15 | 1350 | 60 | 12 | 0 | 500-850 | 1500 | 88.5% |
| 12-16 | 1350 | 75 | 12 | 0 | 500-850 | 1500 | 86% |

### Example #2

### Reaction With Propane

In a typical case with C₃H₈, the reactor is preheated to the desired temperature with flowing N₂ (from liquid nitrogen tank). The bed materials (150 to 300 g) are ground and sieved particles from the previous runs with a size between 300-800 microns. The hydrocarbon (C₃H₈) from liquid propane tank along with diluting gas nitrogen was regulated through two mass flow controllers. The inlet pressure is maintained at 30 Psi and the amount of propane is monitored using an electronic scale. The gas mixture (the concentration was determined by experiment design) was introduced into the reactor when the reactor reaches the desired temperature. Once the run time is reached, the reaction is stopped and the reactor is cooled to room temperature and break down to extract the products. Since the density of the sample has a great impact on the mechanical strength of the mechanical properties, therefore, it was used as initial measure to monitor the process. In addition, the dimension or weight of the samples, the weight of carbon media left in the reactor (the size of the fluidized bed), the weight of the media withdrawn was measured.

Table 2 shows the example runs conducted. Specific details of the experiment associated with each run. Pure pyrolytic carbon samples were prepared as controls for the properties comparison. Carbon nanofiber reinforced pyrolytic carbon coating samples were prepared to compare the micro-scale structures and the macroscopic properties respectively. As an option, Fe(CO)₃ was introduced as a catalyst for comparison and not obvious enhancement for the conversion has observed.

TABLE 2
Experimental conditions and conversion for propane pyrolysis

| | | | % Propane | | | |
|---|---|---|---|---|---|---|
| Run | | Time | in | | Fe(CO)₃ | |
| Number | T (° C.) | (min) | Nitrogen | Bed (g) | (mg/min) | Final Bed (g) |
| 1 | 1000 | 240 | 20 | 200 | 0 | 210 |
| 2 | 1300 | 165 | 25 | 200 | 0 | 300 |
| 3 | 1300 | 180 | 25 | 200 | 0 | 400 |
| 4 | 1350 | 180 | 25 | 250 | 0 | 805 |
| 5 | 1350 | 310 | 25 | 200 | 0 | 1340 |
| 6 | 1325 | 300 | 25 | 150 | 0-2 | 1013 |
| 7 | 1200 | 240 | 25 | 200 | 0-4 | 270 |
| 8 | 1350 | 120 | 25 | 200 | 0 | 700 |
| 9 | 1325 | 185 | 25 | 150 | 2 | 750 |
| 10 | 1100 | 150 | 25 | 150 | 1 | |
| 11 | 1000 | 60 | 25 | 200 | 2 | |
| 12 | 980 | 60 | 15 | 200 | 4 | |
| 13 | 1300 | 75 | 40 | 200 | 0 | 300 |
| 14 | 1350 | 240 | 40 | 180 | 0 | 217 |
| 15 | 1325 | 240 | 50 | 180 | 0 | 750 |
| 16 | 1350 | 240 | 50 | 180 | 0.5 | 1012 |
| 17 | 1350 | 24 | 25 | 180 | 0 | 720 |
| 18 | 1350 | | 50 | 180 | 1 | 1040 |
| 19 | 1350 | 300 | 60 | 150 | 0 | 525 |
| 20 | 1350 | 300 | 60 | 150 | 2 | 1130 |
| 21 | 1350 | 60 | 60 | 150 | 3 | |
| 22 | 1350 | 300 | 60 | 150 | 0 | 950 |
| 23 | 1350 | 100 | 60 | 150 | 3 | 278 |
| 24 | 1350 | 156 | 60 | 150 | 2 | 970 |
| 25-6 | 1350 | 150 | 6/15 | 200 g | 0 | 580 |
| 26-8 | 1350 | 60 | 100 | 330 | 0 | 684 |

### Example #3

### Partial Oxidation

In partial oxidation reaction, 500 grams of nanostructured carbon particles (500-1000 um) were preheated in nitrogen to 1200° C. Premixed gas: CH₄(5.4 LPM) and air (15 LPM) were introduced according to the stoichometric ratio

2CH₄+O₂→4H₂+2CO

After reaction the carbon particles in the reactor were weighted to be 497 gram without significant weight change. Therefore, the carbon particles are used as catalyst for the reaction. This approach has the advantage of catalyst self generating and self activation by maitain the fuel/air ratio during the reaction.

Although the invention has been described in terms of the preferred embodiments which constitute the best mode presently known to the inventors for carrying out the invention, it should understood that various changes and modifications as would be obvious to one having the

What is claimed is:

1. A method for manufacturing nanostructured carbon from hydrocarbons, the method comprising the steps of:
decomposing hydrocarbons thermally using a first carbon particles as substrates and catalyst in a reactor;
separating a solid phase carbon from a gas phase;
recovering a hydrogen-containing gas from the reactor; and
withdrawing a second carbon particles from the reactor

2. The method as in claim 1, wherein the first carbon particles are nanostructured carbon particles.

3. The method as in claim 1, wherein the first carbon particles are in the range of 0.1 to 2 mm.

4. The method as in claim 1, wherein the first carbon particles are in the range of 0.3 to 0.5 mm.

5. The method as in claim 1, wherein the second carbon particles are nanostructured carbon particles.

6. The method as in claim 1, wherein the second carbon particles are in the range of 0.1 to 5 mm.

7. The method as in claim 1, wherein the hydrogen-containing gas is free from carbon oxide.

8. The method as in claim 1, wherein the second carbon particles have the purity of at least 95%.

9. The method as in claim 1, wherein the reactor is at a temperature of from approximately 1000° C. to approximately 2800° C.

10. The method as in claim 1, where in the reactor has a pressure range of approximately 0.1 to approximately 2000 psi.

11. The method as in claim 1, further comprising the steps of:
grinding certain amount of the second carbon particles: and
reintroducing into the reactor to balance the total bed surface area.

12. System for manufacturing nanostructured carbon according to claim 1, the system comprising:
a spouted bed reactor chamber;
a heating system
a thermal insulation system;
a chemical introducing system;
a gas and solid separation system;
a gas analysis system;
a plurality of introducing ports
a particle feeding system;
a particle withdrawing system; and
an internal grinding system.
a preheating and heat recovery system
a monitor system for the structure of carbon particles.

13. The system as in claim 12, wherein the heating system is chosen from the group comprising:
electrical resistive heating;
RF inducting heating;
microwave heating;
thermal plasma heating;
combustion heating by a self-heating using hydrogen, un-reacted hydrocarbon, carbon
particles, or other fuels;
solar energy; and
nuclear energy heating.

14. A composition of nanostructured carbon, characterized in that comprising:
a density from 1.7 g/cc to 2.3 g/cc;
a lattice spacing from 2.37 A to 2.8 A;
a crystalline size from 10 A to 200 A; and

15. The composition of nanostructured carbon as in claim 14, wherein the nanostructured carbon is for use as catalyst in hydrocarbon reactions.

16. The composition of nanostructured carbon as in claim 15, wherein the hydrocarbon reaction is decomposition of hydrocarbons.

17. The composition of nanostructured carbon as in claim 15, wherein the hydrocarbon reaction is partial oxidation of hydrocarbons.

18. The composition of nanostructured carbon as in claim 15, wherein the hydrocarbon reaction is hydrogenation of hydrocarbons.

19. The composition of nanostructured carbon as in claim 15, wherein the hydrocarbon reaction is dehydrogenation of hydrocarbons.

20. The composition of nanostructured carbon as in claim 14, wherein the nanostructured carbon is used as solid fuel of direct carbon fuel cells.

21. The composition of nanostructured carbon as in claim 14, wherein the nanostructured carbon is used as anode of lithium ion battery.

22. The composition of nanostructured carbon as in claim 14, wherein the nanostructured carbon is used in an electrochemical device.

23. The composition of nanostructured carbon as in claim 14, wherein the nanostructured carbon is used as fillers or components of an implantable medical device.
